(19) United States
(12) Patent Application Publication
(10) Pub. No.: US 2023/0277383 A1
(43) Pub. Date: Sep. 7, 2023

(54) SURGICAL DRAIN PROTECTIVE CUSHION

(71) Applicant: Susie Schimpf, Willard, MO (US)

(72) Inventor: Susie Schimpf, Willard, MO (US)

(21) Appl. No.: 17/684,516

(22) Filed: Mar. 2, 2022

(51) Int. Cl. A61F 13/00; A61L 15/24; A61L 15/26

(52) U.S. Cl. CPC A61F 13/00068; A61F 13/00017; A61F 13/00021; A61L 15/24; A61L 15/26

(57) ABSTRACT

A topical cushion configured to be worn in proximity to a surgical site of a patient. Such a topical cushion may be further configured to allow free movement at or near the surgical site, while preventing painful or otherwise unwanted contact.

### CROSS-REFERENCES TO RELATED APPLICATIONS

Not Applicable.

### FIELD OF INVENTION

The present invention relates generally to a low-impact topical medical device. More particularly, the present invention is related to a protective cushion and/or support device for use by patients having surgical drains and/or implanted ports.

### BACKGROUND OF THE INVENTION

Surgical drains are commonly used in patient's recovering from surgical procedures to collect and remove fluid that can build up within a body cavity. This typically requires that a surgical tube be left in place, passing through an incision site, for an extended period, commonly days to weeks. As a result, a great deal of care is required to protect surgical drains from being knocked around or dislodged during normal movement and/or activities, as this can be painful, can risk irritation or infection, or may cause the drain to become unseated or otherwise require replacement of the device.

For at least these reasons, there exists a need for an improved means of protecting surgical drains and similar devices from painful or otherwise unwanted contact.

### SUMMARY OF THE INVENTION

This Summary is provided to introduce a selection of concepts in a simplified form that is further described below in the Detailed Description. This Summary is not intended to identify key features or essential features of the claimed subject matter, nor is it intended to be used as an aid in determining the scope of the claimed subject matter. The present invention is defined by the claims.

Embodiments of the present invention may comprise a topical cushion configured to be worn in proximity to a surgical site of a patient. Such a topical cushion may be further configured to allow free movement at or near the surgical site, while preventing painful or otherwise unwanted contact such as impact, torsion, and/or compression. Some embodiments may be held in place via a compression band, wherein such compression band is configured to wrap around some portion of the patient's anatomy.

### BRIEF DESCRIPTION OF THE DRAWINGS

The present invention is described in detail below with reference to the attached drawing figures, wherein:

FIG. 1 provides a prospective view of an exemplary embodiment in accordance with aspects of the present invention.

FIG. 2 provides a view of an exemplary embodiment as it may appear in use in accordance with aspects of the present invention.

FIG. 3 provides a top view of an exemplary embodiment in accordance with aspects of the present invention.

### DETAILED DESCRIPTION OF THE INVENTION

Some embodiments of the present invention are described with specificity herein to meet statutory requirements. However, the scope of the invention is not intended to be defined by the description itself. The claimed subject matter may be embodied as to include different features, elements, components, steps, or combinations of steps, similar to those described herein, and in combination with other existing or future technologies. Moreover, although the term “step” might be used to connote different elements of the methods employed, this term should not be interpreted as implying any particular order among or between various steps herein disclosed unless and except the order of individual steps is explicitly described or required.

Embodiments of the present invention may comprise a topical cushion configured to be worn in proximity to a surgical site of a patient. As used herein, the terms “wearer”, “user”, “patient”, and the like may be used interchangeably to refer to the party making use of the present invention. As used herein, the term “surgical site” is used broadly to refer to a location on a patient's skin corresponding to the location of a surgical incision and/or the placement of a medical device or the like for which one having skill in the art may recognize increased protection against unwanted contact may be desirable. Surgical sites may include but are not limited to the location of a Jackson-Pratt drain, a Penrose drain, and implanted port, a catheter of any kind, and the like.

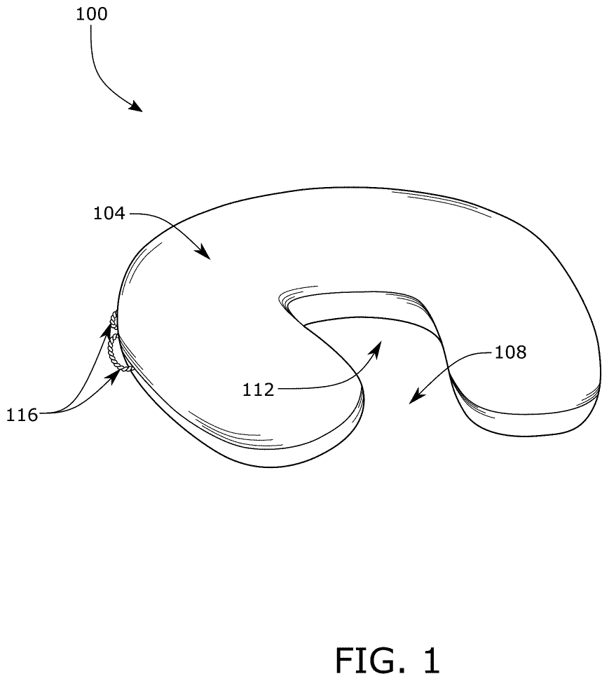

In some embodiments a topical cushion may be configured to allow free movement at or near the surgical site, while preventing painful or otherwise unwanted contact such as impact, torsion, and/or compression. FIG. 1 provides a prospective view 100 of an exemplary embodiment in accordance with aspects of the present invention. As depicted, cushion 104 has a generally “U” shaped profile, with open end 108 and enclosed area 112. Enclosed area 112 may be configured to be placed around a surgical site (not pictured) such that cushion 104 may provide additional protection against unwanted contact. Additionally, enclosed area 112 may be configured to provide optimal balance of protection to freedom of movement around the surgical site. Open end 108 may be configured such that surgical tubing, catheters, and the like may reach the surgical site without impedance. Loops 116 may be configured to receive a belt and/or compression band for use in securing cushion 104 to a user.

Embodiments may comprise various cushion materials having a preferred balance of firmness for protection and flexibility and softness for comfort. Examples of materials that might be used include, but are not limited to, injection molded Ethylene-vinyl acetate (EVA) foam, high-density Polyurethane, and the like or a combination thereof.

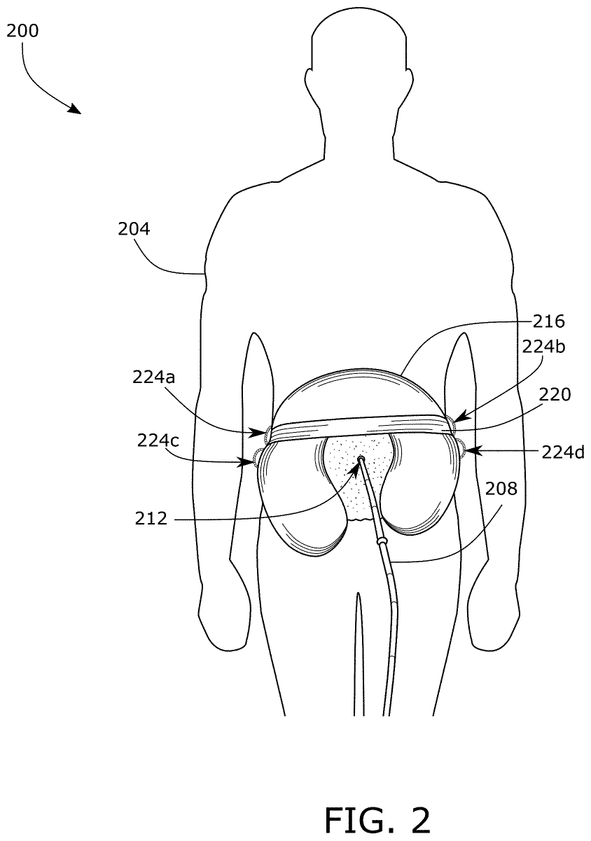

FIG. 2 provides a view 200 of an exemplary embodiment as it may appear in use in accordance with aspects of the present invention. As depicted, patient 204 has surgical drain 208 installed at surgical site 212. Cushion 216 is secured in proximity to surgical site 212 via compression band 220. As depicted, compression band 220 is configured to wrap around patient 204 at the torso. One having skill in the art will recognize that, for different surgical site locations such a compression band may be configured to wrap around some other corresponding portion of the patient's anatomy. Compression band 220 is depicted running through upper loops 224a-b, which aid in securing compression band 220 to cushion 216. In a different configuration, compression band 220 may be run through lower loops 224c-d or some combination of upper and lower loops 224a-d depending on the desired positioning of cushion 216 and the comfort of the wearer.

Compression band 220 may comprise a material having sufficient tensile stretch to secure cushion 216 in place, while flexible and breathable enough to permit wearing for extended periods. Such materials may include, but are not limited to, woven fabrics comprised of cotton, linen, hemp, and the like. One having skill in the art will recognize various materials having the desired characteristics may be applied to this purpose. In an alternative embodiment, cushion 216 may be secured directly to the body of patient 204 via an adhesive (not shown) in lieu of or in addition to compression band 220.

Returning to FIG. 2, the generally “U” shaped profile of cushion 216 provides protection to the area surrounding surgical site 212 while enabling surgical drain 208 to freely reach surgical site 212 unincumbered.

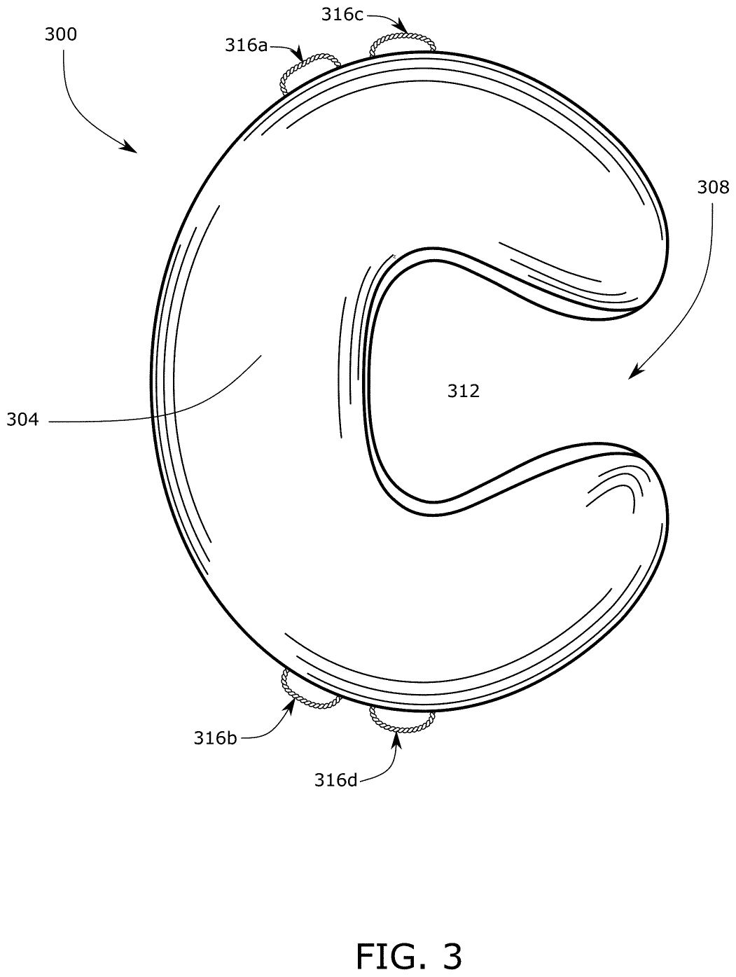

FIG. 3 provides a top view 300 of an exemplary embodiment in accordance with aspects of the present invention. Cushion 304 has an open end 308 and enclosed area 312. Upper set of loops 316a-b and lower set of loops 316c-d may be configured to receive a belt and/or compression band for use in securing cushion 304 to a user.

Embodiments of the present invention have been described, as required by statute, to be illustrative, but should not be interpreted to be restrictive. One having skill in the art will recognize that many different arrangements of the various components depicted are possible without departing from the scope of the claims below, as well as arrangements including components not explicitly shown.

One having skill in the art will understand that certain combinations and/or sub-combinations of elements and features are of utility and may be employed without reference to other combinations and/or sub-combinations and are contemplated within the scope of the claims. Not all steps listed in the various figures need be carried out in the specific order described.

What is claimed is:

1. An apparatus comprising:
a cushion having a “U” shaped profile,
wherein the cushion is configured to be placed around a surgical site of a patient.

2. The apparatus of independent claim 1, further comprising:
a compression band configured to secure the cushion to a body of the patient by wrapping around a portion of the body of the patient.

3. The apparatus of independent claim 1, further comprising:
one or more sets of loops configured to receive the compression band.

4. The apparatus of independent claim 1, wherein:
the cushion comprises at least one of an injection molded Ethylene-vinyl acetate (EVA) foam and a high-density Polyurethane.
